(19) United States
(12) Patent Application Publication
(10) Pub. No.: US 2006/0137696 A1
(43) Pub. Date: Jun. 29, 2006

(54) Zone-based pediatric and veterinary dosing system

(76) Inventor: James B. Broselow, Hickory, NC (US)

Correspondence Address: Adams Evans P.A., 2180 Two Wachovia Center, 301 S. Tryon Street, Charlotte, NC, 28282, US

(21) Appl. No.: 11/025,404

(22) Filed: Dec. 29, 2004

(51) Int. Cl. A61B 19/00

(52) U.S. Cl. 128/898

(57) ABSTRACT

A method of determining proper medical treatment values for a patient, including the steps of a method of determining proper medical treatment values for a patient, including the steps of determining a body weight of a patient. The weight can be determined directly or by reference to a weight-correlated measurement, such as length. A medical administration/preparation (“MAP”) guide is provided for each of a plurality of medical treatments, each MAP guide having a plurality of treatment values provided thereon correlated to a respective plurality of patient weight zones. The treatment value for the patient is determined by reference to the MAP guide. The medical treatment is administered as indicated on the MAP guide for the patient based on the weight zone of the patient as determined by the weight-determining step.

### TECHNICAL FIELD AND BACKGROUND OF THE INVENTION

This invention relates to a zone-based dosing system designed to aid in the practice of pediatric and veterinary medicine. Medication dosing is simplified by the development of color-coded dosing zones based on patient weight or length, depending on the circumstances. When weights are readily available, such as in offices, clinics, or hospitals, weight is used as the universal dosing value. When weight is not available, the child's length can be used to determine his “color.” Once the proper dosing “color” has been established, the color remains the same for all medication dosing so long as the child's weight or length remain within the range of weights or lengths applicable to that color.

Length-based dosing will often be more appropriate for emergencies where weight is frequently not known. This system can also be used for home dosing of medications where accurate weights are also usually not available. All patients within a given zone would be given the same dose range of medication.

The invention also has application for determination of other physical treatment values, such as the proper size of pediatric equipment, ventilator settings, infusion devices, etc. The zones would be labeled with the name of the color for use by those who are color blind.

In adult medicine the dosing of medications and selection of appropriately-sized equipment is straightforward. A cardiac arrest, for instance, can be treated by giving “an amp” of epinephrine or bicarbonate. Likewise the initial dose of atropine for an adult may be given as 0.5 mg of atropine for bradycardia. This dosage may be given safely to a broad spectrum of patients. Similarly an adult with a fever can take two aspirins or two Tylenol tablets and feel comfortable that he has taken an appropriate dose. An adult patient can be intubated with one of only a few sizes of endotracheal tubes, and it is not difficult to learn which size tube to use based on experience in dealing with differently-sized adults. This is obviously not the case in pediatrics. Children's sizes vary tremendously from the smallest premature infant to a large pre-pubital child. There are more than ten endotracheal sizes that may be appropriate for different size pediatric patients. Medications are most commonly dosed according to patient weight and dosages are expressed in ranges of mg/kg. To arrive at an appropriate dose one must know the patient's weight, the formula in mg/kg, and the concentrations of the drug which are available. Then, mathematical calculations must be done to arrive at the appropriate dosage. Using weight to dose becomes problematic in circumstances where weight is not known. This is true in emergency situations such as pediatric cardiac arrests, seizures, and trauma situations. In addition, it is quite difficult to remember formulas and do mathematical computations accurately in the excitement of a pediatric resuscitation. It may take even a seasoned clinician years to get used to the nuances of dosages and equipment sizes and equipment settings in children. Even with experience, few clinicians are really comfortable with giving children drugs such as IV sedatives, pain medications, paralyzing agents, to name only a few.

The everyday practice of pediatrics is constantly interrupted by the question of how much of a given drug should be given to a particular pediatric patient. Physicians such as orthopedists and surgeons spend a significant amount of time looking up dosages. Frequently sufficient time is not taken, or inadvertent mistakes are made, or the dosages are determined by educated guess, giving erratic and inconsistent dosages of many medications to children. In emergencies this problem is magnified because there may not be time to look up the dosage.

This also creates problems for nurses who are responsible for giving the proper dosage, yet may not have the time to verify an order under these circumstances. In addition, non-physicians may be called upon to dose children under emergency situations such as a paramedic treating a child who is seizing with IV or rectal Valium, or cardiac arrest. Even an emergency physician may feel uncomfortable with IV demerol for a child with a broken leg, in part due to concern about giving the proper dose to give pain relief without developing respiratory insufficiency. The resulting dilemma tends to lead to underutilization of pain medications and undue suffering to the pediatric patient.

This problem also impacts the field of medical education where each year there is an influx of first year residents who are called upon to dose children in hospitals and clinics. Unfortunately, even the best of childrens′ hospitals and medical centers report their share of dosing mistakes, occasionally with tragic outcomes. This problem is not unique to the medical community. The lay public has great concerns about giving medications to children. Studies have shown that in the common scenario of a child with a fever, dosages of acetaminophen are frequently inadequate and ineffective, contributing to marked parental anxiety. Inadequate dosing may also contribute to unnecessary trips to emergency departments, since a fever that doesn't come down with antipyretics is a common reason for an ED visit. Deaths have been reported resulting from inadvertant overdosing of fever medication at home. Even physicians are concerned about how much of such seemingly innocuous agents as decongestants and cold preparations to give their own children or grandchildren. If they don't practice pediatrics, they may find themselves calling a colleague to ask how much Dimetapp to give their one year old child.

In addition, the FDA has been hesitant to give dosing information to the public for young children. The bottle label tells them to call their doctor for instructions. This is not, however, a practical solution to this problem since they may not have a doctor or he may be unavailable at that time. In addition, many people do not feel comfortable calling under those circumstances, and find themselves giving medication anyway, perhaps asking a grandmother how much, or just taking a guess at the amount.

This entire problem is compounded by the large number of people who are both professionally and personally called upon to give medications to children. At the top are pediatricians who are quite adept at dosing pediatric patients, except possibly in the area of pediatric emergencies. However, many other medical specialties are called upon to dose children and lack the expertise of pediatricians. At best it can be very time-consuming for physicians and nurses to find the proper dose. At worse, mistakes are made. In addition there are paramedics, nurse practitioners, physician's assistants, health department nurses, daycare center personnel, teachers and parents, all whom may be called upon to decide how much medication a child should take.

In the area of pediatric emergency medicine this problem has been previously addressed by the development of a tape measure, the Broselow Tape, which allows the dosage of medications and equipment sizes to be determined from a direct length measurement of the patient. The tape is disclosed in U.S. Pat. No. 4,713,888. This has been found to be useful since it makes unnecessary the estimation of weight in an emergency, memorization of infrequently used formulas and the necessity to do mathematical calculations under duress. The equipment zones on the Broselow tape are color-coded to allow storage and access of emergency equipment by color, allowing more rapid access and easier restocking. For these reasons, the Broselow tape is now well accepted and widely used.

This application relates to the development of a voluntary, provider driven zone-based pediatric dosing system based on the concept of developing dosing zones not only for equipment but for drugs as well. These zones would be designed so that all children within the zone could be given the same dosage instructions specific for that drug. The dose could be a single fixed amount such as for liquid Tylenol, or could be a range for titrating IV medications such as demerol or Versed. For example, assume that a child presents to an emergency department with a painful fracture. His color could be obtained from a Broselow tape while lying on the stretcher. If he were in the “blue” zone, he would be given the “blue dose” of demerol. The “blue dose might say “give 10 mgs of Demerol IV every 3 to 5 minutes until pain relieved.” If the child were to become unduly sedated, he could be given the “blue dose” of Narcan. It seems likely that a child would more readily be given pain medications if dosing were not an issue.

A similar example could be given for a child who needs sedation for a CT scan. It would be a simple matter for the radiologist to order an appropriate dose without having to confer with the hospital pharmacy or a pediatrician. Surgery procedures could also be indexed by the dosing color. For instance, the procedures could be identified as “three blue cases and two red cases this morning in surgery.”

This would allow the drugs and equipment for each case to be prepared prior to surgery. The pre-op medications and pain medications as well as anesthetics would all be indexed to this color. The maintenance fluids during surgery could be given by setting the infuser to the proper color.

Likewise in the field of Veterinary medicine, even more than in human medicine where many dosages are standardized, individual calculations are required for virtually all patients. Not only must the Veterinarian be able to determine the dose in mgs similar to how a doctor presently would give an order, but he frequently must calculate how that mg can be obtained from liquid containers where the dose is presented in various confusing forms. E.g 10 ml vial, containing 300 mgs of a drug representing a concentration of 30 mgs/ml. If his calculated dose is, for instance, 45 mgs of the drug, he would set up a ratio and cross multiply to determine that he needed 1.5 ml of that drug.

In human medicine frequently this calculation is done by a second person, (nurse or pharmacist) who help administer the medication. A veterinarian, however, must simultaneously play multiple roles, ie be the surgeon plus give the anesthesia.

He also is the emergency doctor, writes nutritional orders and prescriptions for out patient medications. All of these processes could be rapidly expedited with a color-coded system. The simplicity of this system would allow him to train relatively inexperienced assistants to prepare drugs for administration. An additional advantage is the fact the dogs and cats wear collars where their “Dosing color” could be attached. If they arrived in an emergency veterinary clinic that color could expedite emergency care. In addition, the color could be used to recognize actual or ideal weight for the dog or cat that could be correlated with feeding instructions in written form or affixed to the package of pet food. A color-coded cup could give ideal body weight feeding related to the dog or cat's “preferred weight color” rather than the actual weight.

The system would be implemented through the use of a System Access Guide which contains a Medication Administration/Preparation (MAP) page for each medication. The MAP page contains basic information for the dosing of a medication. The dosage of medication in milligrams and the volume of drug in milliliters for the patient is obtained by referring to the patient's color on the MAP page. The clinician can choose to dose utilizing the standard dose or he/she may elect to bypass the system and choose a different dose for his/her patient.

For example, a physician is caring for a child in the emergency room and needs to know what dose of Atropine to give the child. The physician will determine the child's color using the Broselow Tape disclosed in U.S. Pat. No. 4,713,888 and then go to the System Access Guide, look up the MAP for Atropine and then dose the child according to the color on the MAP. The physician may also decide to raise or lower the dosage as he/she sees fit. This system provides a failsafe mechanism for dosing children with potentially toxic drugs.

A similar system is disclosed in Broselow U.S. Pat. No. 6,132,416. That system incorporated the color coding into the actual dosing vehicle such as color-coded syringes used particularly in the area of pre-hospital treatment of pediatric emergencies. Likewise, the colors were incorporated into dosing devices for the public such as color-coded cups, syringes, etc. for use with OTC medications. However, that patent was a pharmacy driven system instead of a provider driven system. In addition, while it provided a failsafe system it still had some disadvantages that have been remedied by this invention. For example, one disadvantage is the fact that each color coded syringe had to match a specific medication. If the wrong syringe was used, the result could be an overdose or perhaps not enough medication being administered. Thus, each syringe must be carefully marked and selected for each type of medication. This could cause confusion in an emergency situation or worse yet for a parent who is administering medication to their child. This is solved by this invention. By using the MAP pages a dosing amount is given. Thus, it does not matter what type of syringe or measuring cup is used so long as it is capable of measuring the amount needed. For example, suppose a parent must administer their child five different drugs each having a different dosing amount. Using the system disclosed in U.S. Pat. No. 6,132,416 would require the parent to make sure that he/she used the proper syringe or measuring cup when dosing their child because a blue dosage of one medication may not be the same as the blue dosage of another medication. This could be very confusing to a parent. This invention simplifies this process by providing the parent with actual dosages for their child. Thus, the parent could use one syringe or one measuring cup to dose their child. The parent would simply look at the MAP, find her child's color and then give the child that dosage. This makes the differences between blue dosages of different medications irrelevant because the parent is actually measuring the dosage for each medication based on the blue dosage indicated on the MAP.

The simplicity of the system should encourage both the public and professionals to turn to color-coded sources for dosing information. Injury and accident instructions as well as aftercare instructions could all be related to the child's “color.” It would help people focus on the different needs of children of different sizes in a simple and visually graphic way. By incorporating injury and accident prevention information into these materials, they would serve as an important reminder to health care professionals to reinforce these important messages with each patient encounter. When one looks at the tremendous variety of situations in which children are given medications, color coding is an effective way of enhancing reliability, safety and efficacy.

### SUMMARY OF THE INVENTION

Therefore, it is an object of the invention to provide a drug dosing system which reduces the possibility of incorrect dosing;

It is another object of the invention to provide a drug dosing system which does not require reformulation of existing drug concentrations or potency;

It is another object of the invention to provide a drug dosing system which is color-coded so as to be usable by individuals who do not read, or who cannot read a particular language in which dosing instructions would ordinarily be given;

It is another object of the invention to provide a drug dosing system which is color-coded so as to be usable by veterinarians to treat animals.

It is another object of the invention to provide a drug dosing system which insures by code, that the appropriate amount of drug is dispensed for a given color-correlated drug concentration or potency.

It is another object of the invention to provide a drug dosing system which correlates weight-related values indicative of a proper dosage with a predetermined, arbitrary color.

These and other objects of the invention are achieved by a method of determining proper medical treatment values for a patient, including the steps of determining a body weight of a patient. The weight can be determined directly or by reference to a weight-correlated measurement, such as length. A medical administration/preparation (“MAP”) guide is provided for each of a plurality of medical treatments, each MAP guide having a plurality of treatment values provided thereon correlated to a respective plurality of patient weight zones. The treatment value for the patient is determined by reference to the MAP guide. The medical treatment is administered as indicated on the MAP guide for the patient based on the weight zone of the patient as determined by the weight-determining step.

According to one preferred embodiment of the invention, the treatment value comprises a medication dosage.

According to another preferred embodiment of the invention, the MAP guide includes information indicating the medication concentration and the means of administration to the patient.

According to yet another preferred embodiment of the invention, the treatment value comprises a medical equipment size.

According to yet another preferred embodiment of the invention, the treatment value comprises a difibrillation dosage.

According to another preferred embodiment of the invention, the MAP guide includes thereon preparation and administration instructions.

According to yet another preferred embodiment of the invention, the MAP guide includes minimum and maximum treatment values.

According to yet another preferred embodiment of the invention, the MAP guide includes a flow chart of adminstration steps.

According to yet another preferred embodiment of the invention, the weight zones are identified by different colors, and the plurality of treatment values of the MAP guide are identified by respective colors corresponding to the colors of the plurality of weight zones.

According to yet another preferred embodiment of the invention, a method of determining proper medical treatment values for a patient is provided, and includes the steps of comprising the steps of grouping a plurality of patient body weights into a plurality of distinct, color-specific weight zones, and providing a medical administration/preparation (“MAP”) guide for each of a plurality of medical treatments. Each MAP guide has a plurality of treatment values provided thereon identified by color in a manner corresponding to the plurality of color-specific weight zones. The patient is weighed, and the treatment value for the patient is determined by selecting the color-specific weight zone of MAP guide corresponding to the weight of the patient. The medical treatment is administered as indicated on the MAP quide for the patient based on the weight of the patient as determined by the weighing step.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 shows a zoned-based dosing system using a System Access Guide and a MAP;

FIG. 2 shows the front side of a MAP for the drug Atropine;

FIG. 3 shows the reverse side of a MAP page for the drug Atropine;

FIG. 4 shows a front side of a MAP page for Lidocane;

FIG. 5 shows a reverse side of a MAP page for Lidocane;

FIG. 6 shows a flow chart, contained on a MAP page, for the administration of IV Pentobarbital; and

FIG. 7 shows a MAP for equipment and cardioversion/defibrilation.

### DESCRIPTION OF THE PREFERRED EMBODIMENT AND BEST MODE

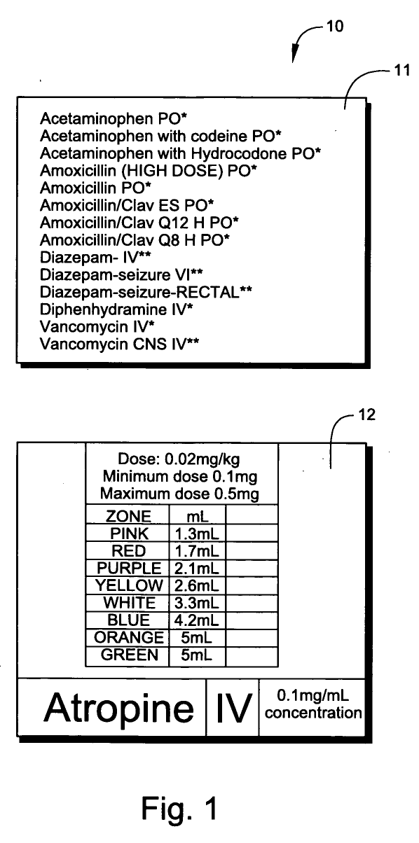

Referring now specifically to the drawings, a zone-based dosing system according to an embodiment of the invention is shown in FIG. 1 at reference numeral 10. The system has application in both human and veterinary medicine. The system 10 includes a System Access Guide 11 and a MAP page 12. The System Access Guide 11 provides access to the MAP pages 12. It contains the standard drug doses which can be ordered for a patient. The medication is ordered by writing the dose and indication. The dosage of medication in milligrams and the volume of drug in milliliters for the patient is obtained by referring to the patient's color on the MAP page 12. The System Access Guide 11 comprises desk reference and pocket guides. The MAP components 12 comprises MAP books and software for all areas and pharmacy MAP cards for ALS areas. The system is a voluntary, provider driven system as opposed to a pharmacy driven system as are most standard dosing systems. The system uses a correlation such as disclosed in the Broselow U.S. Pat. No. 4,713,888, and a given color is selected so that, for example, a dosage for a patient of a given weight or body length is always, for example, blue.

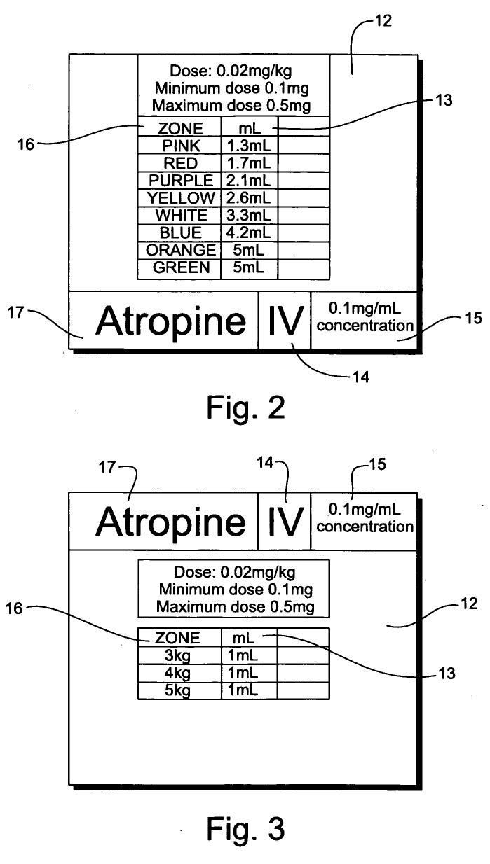

Referring to FIGS. 2 and 3, the MAP 12 is a two sided sheet. As shown in FIG. 2, side one contains the name of the drug 17, the pre-calculated dosage 13, the color zone 16, the route 14, and the concentration 15. As shown in FIG. 3, side two contains the same information for the 3, 4, and 5 kg patients.

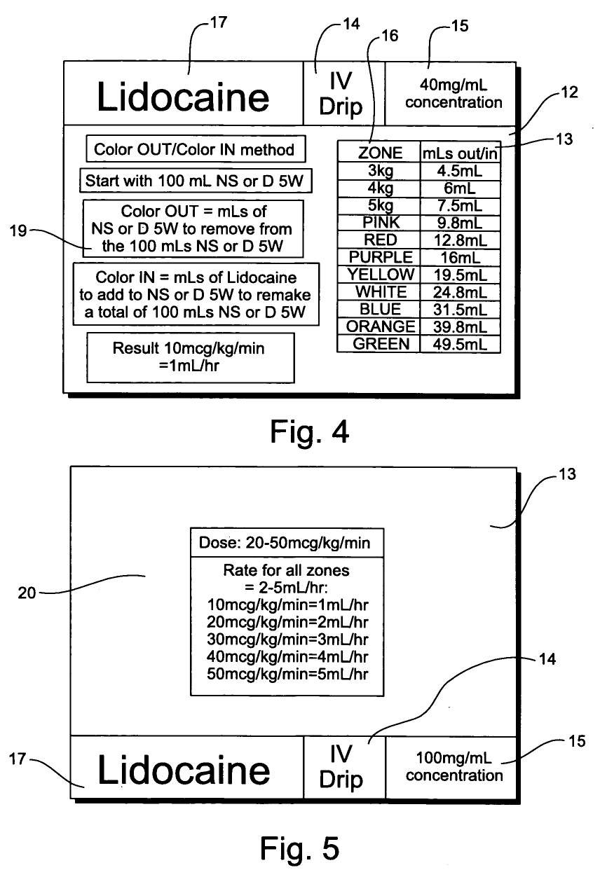

Referring to FIGS. 4 and 5, the MAP 12 can also contain detailed preparation 19 and administration instructions 20. FIG. 4 shows side one of a MAP 12 for Lidocaine. Side one contains the name of the drug 17, the pre-calculated dosage 13, the color zone 16, the route 14, the concentration 15, and the detailed preparation 19 for the medication. FIG. 5 shows side two of a Map 12 for Lidocaine. Side two contains the name of the drug 17, the route 14, the concentration 15, and the administration instructions 20 for the medication.

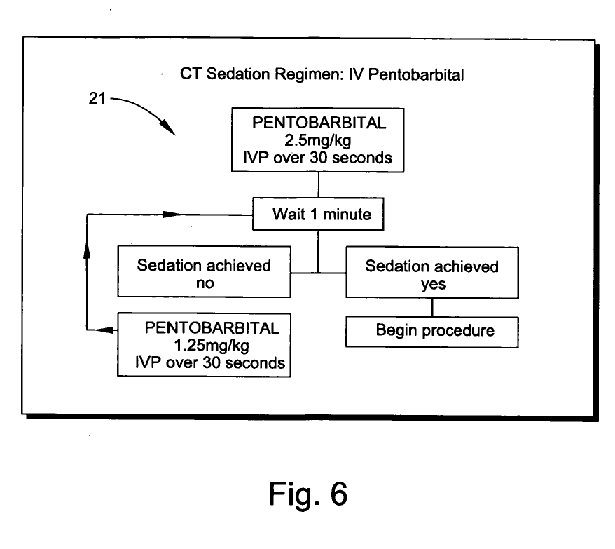

FIG. 6 shows generally a flow diagram 21 for the administration of IV Pentobarbital. The flow diagram 21 can also be incorporated onto a MAP page 12 in situations involving sedation. In this case, the flow diagram 21 details the procedure for sedating a patient with IV Pentobarbital.

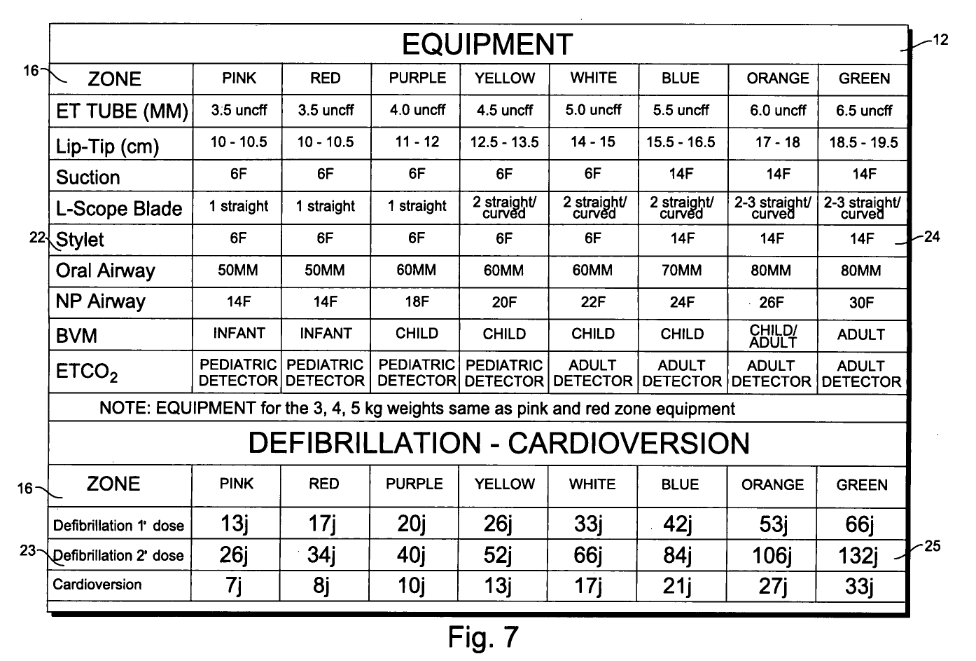

Referring to FIG. 7, the MAP 12 can also be used for Equipment and Cardioversion/Defibrillation. The MAP 12 contains the color zone 16, an equipment list 22, a specification 24 for each piece of equipment, and a Defibrillation dose 23 with the corresponding charge 25.

A zone-based dosing system is described above. Various details of the invention may be changed without departing from its scope. Furthermore, the foregoing description of the preferred embodiment of the invention and the best mode of practicing the invention are provided for the purpose of illustration only and not for the purpose of limitation.

1. A method of determining proper medical treatment values for a patient, comprising the steps of:
(a) grouping a plurality patient body weights into a plurality of patient weight zones identified by distinct colors;
(b) determining a body weight of a patient;
(c) providing a medical administration/preparation (“MAP”) guide for each of a plurality of medical treatments, each MAP guide having a plurality of treatment values provided thereon correlated to the respective plurality of patient weight zones, wherein the plurality of treatment values of the MAP guide are identified by respective colors corresponding to the colors of the plurality of weight zones, and further wherein the plurality of treatment values are based on a universal, consistent dosing unit comprising an actual dosing unit to be provided to the patient;
(d) determining the treatment value for the patient by reference to the MAP guide; and
(e) administering the medical treatment indicated on the MAP guide for the patient based on the weight zone of the patient as determined by the weight-determining step.

2. A method of determining proper medical treatment values for a patient according to claim 1, wherein the treatment value comprises a medication dosage.

3. A method of determining proper medical treatment values for a patient according to claim 2, wherein the MAP guide includes information indicating the medication concentration and the means of administration to the patient.

4. A method of determining proper medical treatment values for a patient according to claim 1, wherein the treatment value comprises a medical equipment size.

5. A method of determining proper medical treatment values for a patient according to claim 4, wherein the treatment value comprises a difibrillation dosage.

6. A method of determining proper medical treatment values for a patient according to claim 1, wherein the MAP guide includes thereon preparation and administration instructions.

7. A method of determining proper medical treatment values for a patient according to claim 1, wherein the MAP guide includes minimum and maximum treatment values.

8. A method of determining proper medical treatment values for a patient according to claim 1, wherein the MAP guide includes a flow chart of administration steps.

9. (canceled)

10. A method of determining proper medical treatment values for a patient, comprising the steps of:
(a) grouping a plurality of patient body weights into a plurality of distinct, color-specific weight zones;
(b) providing a medical administration/preparation (“MAP”) guide for each of a plurality of medical treatments, each MAP guide having a plurality of treatment values provided thereon identified by a distinct color in a manner corresponding to the plurality of color-specific weight zones, wherein the plurality of treatment values of the MAP guide are based on a universal, consistent dosing unit comprising an actual dosing unit to be provided to the patient;
(c) weighing the patient;
(d) determining the treatment value for the patient by selecting the color-specific weight zone of MAP guide corresponding to the weight of the patient; and
(e) administering the medical treatment indicated on the MAP guide for the patient based on the weight of the patient as determined by the weighing step.

11. A method of determining proper medical treatment values for a patient according to claim 10, wherein the color is correlated to mils of a predetermined uniform concentration of a drug.
